(19) United States
(12) Patent Application Publication
(10) Pub. No.: US 2021/0174400 A1
(43) Pub. Date: Jun. 10, 2021

(54) Device for Providing Food Advertisement Based on Food Flavor Through Color Combination and Method Thereof

(71) Applicant: TAIWAN CARBON NANO TECHNOLOGY CORPORATION, MIAOLI COUNTY (TW)

(72) Inventors: Ching-Tung HSU, MIAOLI COUNTY (TW); Chun-Wei SHIH, MIAOLI COUNTY (TW); Chia-Hung LI, MIAOLI COUNTY (TW); Chao-Chieh LIN, MIAOLI COUNTY (TW); Yuan-Shin HUANG, MIAOLI COUNTY (TW); Chun-Hsien TSAI, MIAOLI COUNTY (TW); Chun-Jung TSAI, MIAOLI COUNTY (TW)

(21) Appl. No.: 16/951,717

(22) Filed: Nov. 18, 2020

(30) Foreign Application Priority Data
Dec. 10, 2019 (TW) 108145133

(51) Int. Cl. G06Q 30/02; G01J 3/46

(52) U.S. Cl. CPC G06Q 30/0276; G01J 3/463

(57) ABSTRACT

A device for providing a food advertisement based on food flavors to market a food product, including a carrier and a plurality of color patches provided on the carrier and combined into a color pattern. The color patches are generated according to a colorimetric sensor array sensing the food product, and the colorimetric sensor array includes a plurality of sensing materials for sensing the food product. The sensing materials respectively react with at least one metabolic molecule of the food to change from an initial color to an indication color corresponding to the color patches respectively, so that the color pattern is visually associated with flavor information of the food product correspondingly.

### FIELD OF THE INVENTION

The invention relates to a device for providing a food advertisement and a method thereof, more particularly, to a device for providing a food advertisement through color combinations based on food flavors and a method thereof.

### BACKGROUND OF THE INVENTION

With the popularization of the Internet, social media and mobile phones, the development of the advertising industry has become more diversified. Nowadays, a common advertising manner reach to customers through the Internet services, such as Google and Facebook which serve as a medium to promote products to customers. Furthermore, more companies are collecting online and offline information of consumers, in the hope of further expanding the customer services, and promoting the efficiency of advertising. Online information collection, for example, can be done with browser cookies, while offline information collection is usually more difficult.

Methods for advertising food in the prior art can be found in the U.S. patent publication No. US2013/0282625A1, which discloses a food advertising method, applied to a packaging material for packaging food, the food advertising method includes the steps of: step 1, forming a supplier label, at least a third party label and a commodity name label on the packaging material; step 2, providing, by a food supplier, information about the food on the supplier label, and providing a product name of the food on the product name label; step 3, obtaining, by at least one third-party vendor from the food supplier, a permission to use a third-party label, and step 4, providing, by the third-party vendor, an advertisement on the third-party label.

In practice, the food flavor can only be described in written words, and the words themselves are substantially subjective, unable to objectively and fully get across the exact food flavor. Therefore, even though the effect of a medium can be achieved, the quality of the food may often not live up to the demands of the consumers, and thus the demands of the consumers are not necessarily satisfied.

### SUMMARY OF THE INVENTION

It is a major object of the invention to solve the difficulty in getting across the exact flavor of food to customers and satisfying the demands of the consumers in the conventional food advertisement.

To achieve the above object, the present invention provides a device for providing a food advertisement through color combinations based on food flavors to market food product, including:

a carrier; and

a plurality of color patches provided on the carrier and combined into a color pattern, wherein the color patches are generated according to a colorimetric sensor array sensing the food product, and the colorimetric sensor array includes a plurality of sensing materials for sensing the food product; and wherein each of the plurality of sensing materials reacts with at least one metabolic molecule of the food product respectively, to change from an initial color to an indication color, and wherein the plurality of indication colors correspond to the plurality of color patches respectively, so that the color pattern is visually associated with flavor information of the food product correspondingly.

To achieve the above object, the invention further provides a method for providing a food advertisement through color combinations based on food flavors, including the steps of:

sensing a food product with a colorimetric sensor array, which includes a plurality of sensing materials for sensing the food product, the plurality of sensing materials respectively reacting with at least one metabolic molecule of the food product to change from an initial color to an indication color; and

providing a label including a carrier and a plurality of color patches provided on the carrier, wherein the plurality of color patches corresponding to the indication colors is combined into a color pattern, so that the color pattern is visually associated with flavor information of the food product.

Accordingly, the colorimetric sensor array presents the color patch corresponding to the indication color according to the result of the chemical reaction, and conveys the food flavor to consumers with the color patch, not only providing the information about the food flavor in a more scientific way, but also rendering a better method than written words (i.e., color combinations) for consumers to understand. In other words, the present invention uses the visual presentation of the food flavor as a medium to convey flavor information. For vendors who sell fresh food products which are normally without standard, meaning those fresh food products are inconsistency on quality or nature. By using the colorimetric sensor array of the present invention, the fresh food products to be advertised or sold can be classified in the first place so as to facilitate sales, and consumers are easy to select and purchase based on their own needs.

### BRIEF DESCRIPTION OF THE DRAWINGS

FIG. 1 is a schematic view showing an appearance of an embodiment of the present invention.

FIG. 2 is a schematic view showing the appearance of another embodiment of the present invention.

FIG. 3 is a schematic view showing the appearance of another embodiment of the present invention.

FIG. 4 shows color patterns obtained by a device based on different food products according to an embodiment of the present invention.

### DETAILED DESCRIPTION OF THE PREFERRED EMBODIMENTS

The details and technical aspects of the present invention are now described in conjunction with the accompanying drawings.

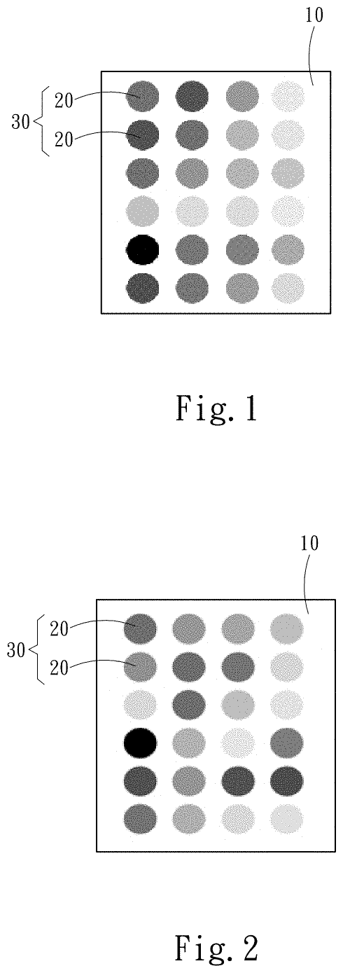

Referring to FIG. 1 and FIG. 2, they are schematic views of the appearances of one embodiment and another one embodiment of the present invention. It discloses a device for providing a food advertisement through color combinations based on food flavors for marketing a food product. The device includes a carrier 10 and a plurality of color patches 20 provided on the carrier 10, wherein the plurality of color patches 20 are combined into a color pattern 30. In this embodiment, the device is a label that can be affixed, attached, or printed to an object which can be the food product or a package of the food product. The plurality of color patches 20 are generated according to a colorimetric sensor array sensing the food product, and the colorimetric sensor array includes a plurality of sensing materials for sensing the food product. The plurality of sensing materials respectively react with at least one metabolic molecule of the food product to change from an initial color to an indication color, wherein the indication colors correspond to the plurality of color patches 20, so that the color pattern 30 is visually associated with a information of the food flavors. In an embodiment, the plurality of color patches 20 and the indication colors have substantially equivalent colors.

In different applications, the plurality of color patches 20 on the carrier 10 can be separately arranged from the colorimetric sensor array, and colors of the plurality of color patches 20 are associated with or equivalent to the indication colors of the sensing materials of the colorimetric sensor array. Alternatively, the colorimetric sensor array may be directly formed with the plurality of color patches 20 on the carrier 10. That is, the plurality of color patches 20 is the plurality of sensing materials of the colorimetric sensor array. In other words, colors of the plurality of color patches 20 are equivalent to the indication colors of the plurality of sensing materials of the colorimetric sensor array. In different applications, the plurality of color patch 20 can display the indication colors of the plurality of sensing material of the colorimetric sensor array in real-time. In one embodiment, the plurality of color patches 20 with similar colors is arranged adjacent to each other, as shown in FIG. 1. In another embodiment, the plurality of color patches 20 with distinct colors is arranged adjacent to each other, as shown in FIG. 2.

In conjunction with the above embodiment, the present invention further discloses a method for providing a food advertisement through color combinations, including the steps of:

step 1: sensing the food product with the colorimetric sensor array that includes a plurality of sensing materials for sensing the food product, the plurality of sensing materials respectively reacting with the at least one metabolic molecule of the food product to change from the initial color to the indication color; and

step 2: providing the label that includes the carrier 10 and a plurality of color patches 20 on the carrier 10, wherein the plurality of color patches 20 corresponding to the indication colors are combined into a color pattern 30, so that the color pattern 30 is visually associated with the flavor information of the food product.

The plurality of sensing materials is selected based on the food product which the plurality of sensing material are applied. The sensing material includes a color generation reagent selected from a group consisting of methyl red, Congo red, bromophenol blue, bromocresol purple, bromocresol green, cresol red, phenol red, thymolphthalein, resazurin, paranitrophenol, bromodaphne blue, thymol blue, neutral red, crystal violet, 4-(4-nitrobenzyl) pyridine, pyrocatechol violet, chlorophenol red, nitrozine yellow, bromophenol red, m-cresol purple, Eriochrome black T, safranine, luciferin, Eosin yellow, Brilliant green, Titan yellow, Victoria blue B, carmine, litmus, curcumin, anthocyanin, alizarin red S, alizarin yellow R, indigo carmine, nile blue A, orange yellow G, Eosin B, 3,3′,5,5′,-tetraiodophenol sulfone phenolphthalein, bromoxylenol blue, phenol blue, disperse orange 25, acridine orange, disperse orange 3, disperse red 1, bromopyrogallol red, diamine diphenyl sulfide maple, aminofluorescein, erythroviolet ammonium urea, 2,6-dichloroindophenol, sodium salts thereof, and mixtures thereof.

In an embodiment, the plurality of sensing material further includes a molecular barrier material selected from a group consisting of octadecanol, polyvinylpyrrolidone, polyvinyl formal, polyvinyl acetate resin, phenolic resin, epoxy resin, polybutene resin, polyethylene glycol, carbon black, carbon nanotubes, graphene, cellulose nanofibers, and silicone compounds. The molecular barrier material is mixed with the color generation reagent to form a sensing pixel; or the molecular barrier material is coated on the color generation reagent to form the sensing pixel. The molecular barrier material can influence a reaction speed of the plurality of sensing material, and the reaction speed can be regulated and controlled by selecting different molecular barrier materials to meet requirements in use.

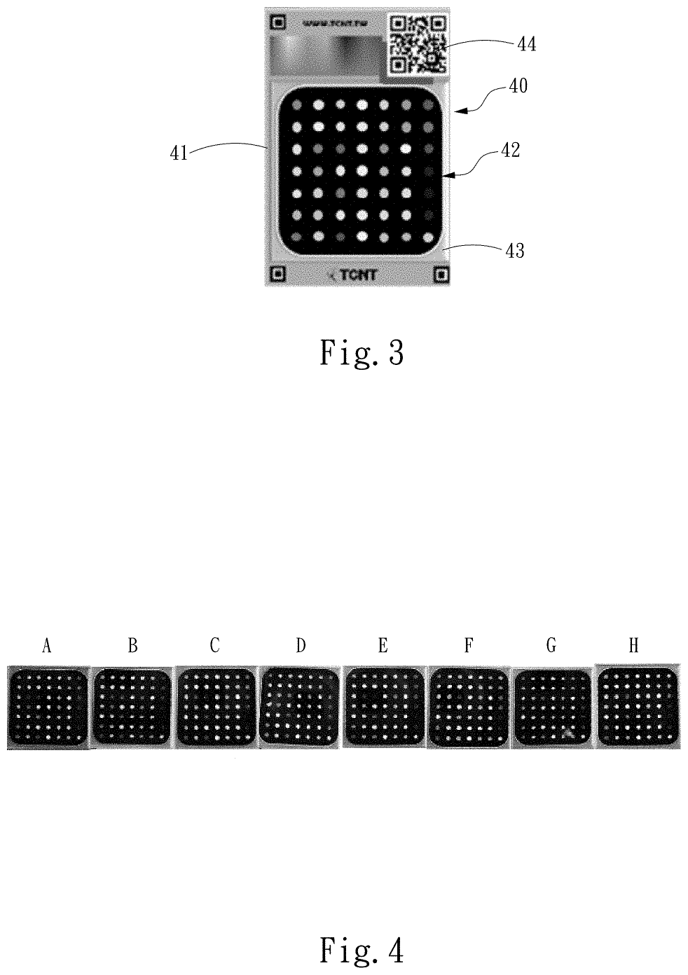

Referring to FIG. 3, a schematic view shows the appearance of another one embodiment of the present invention. In this embodiment, a device 40 provides a food advertisement through color combinations based on food flavors. The device 40 includes a film 41 and a colorimetric sensor array 42 formed on the film 41, and the colorimetric sensor array 42 includes at least one sensing material for sensing the food product. The at least one sensing material reacts with at least one metabolic molecule of the food product to change from an initial color to an indication color. In one embodiment, preferably, the plurality of the sensing materials are provided and formed on the film 41 in a pixel-like manner, such as 7*7 sensing pixels.

To avoid damage from external forces or ultraviolet light, the device 40 further includes a protective layer 43 that covers the colorimetric sensor array 42 to block the ultraviolet light, shield the colorimetric sensor array 42 and prevent the colorimetric sensor array 42 from failure or damage by external forces. In another embodiment, a code 44 is also displayed on the film 41, and the code 44 is associated with the food product and determined based on at least one information of the food product, wherein the at least one information is selected from a group consisting of a history information, a quality information, and an initial appearance; or the code 44 is a string of numbers, a bar code, a QR code, etc.

Referring to FIG. 4, an embodiment of the present invention, the device obtains color patterns according to different food products. In FIG. 4, a color pattern A is corresponding to tap water; a color pattern B is corresponding to deionized water; a color pattern C is corresponding to coffee; a color pattern D is corresponding to rice wine; a color pattern E is corresponding to Pocari Sweat®; a color pattern F is corresponding to the fruit sparkling water; a color pattern G is corresponding to Sprite®; and a color pattern H is corresponding to the red wine. As can be seen from FIG. 4, the colorimetric sensor array will present different color patterns for different food products.

In the present invention, the colorimetric sensor array presents the color patch corresponding to the indication color according to the result of the chemical reaction, and conveys the food flavors to consumers with the color patch, not only providing the information about the food flavor in a more scientific way, and rendering a better method than written words (i.e., color combinations) for consumers to understand. In other words, the present invention presents the food flavors in a visual manner to be a medium for conveying the information of the food flavors. For vendors who sell fresh food products which are normally without standard, meaning those fresh food products are inconsistency on quality or nature. By using the colorimetric sensor array of the present invention, the fresh food products to be advertised or sold can be classified in the first place so as to facilitate sales, and consumers are easy to select and purchase based on their own needs.

What is claimed is:

1. A device for providing a food advertisement through color combinations based on food flavors to market a food product, comprising:
a carrier; and
a plurality of color patches provided on the carrier and combined into a color pattern, wherein the color patches are generated according to a colorimetric sensor array sensing the food product, and the colorimetric sensor array includes a plurality of sensing materials for sensing the food product; and wherein each of the plurality of sensing materials reacts with at least one metabolic molecule of the food product respectively to change from an initial color to an indication color, and wherein the plurality of indication colors correspond to the plurality of color patches respectively, and the color pattern is visually associated with flavor information of the food product correspondingly.

2. The device according to claim 1, wherein colors of the plurality of color patches are substantially equivalent to colors of the plurality of indication colors.

3. The device according to claim 1, wherein the sensing material includes a color generation reagent selected from a group consisting of methyl red, Congo red, bromophenol blue, bromocresol purple, bromocresol green, cresol red, phenol red, thymolphthalein, resazurin, paranitrophenol, bromodaphne blue, thymol blue, neutral red, crystal violet, 4-(4-nitrobenzyl) pyridine, pyrocatechol violet, chlorophenol red, nitrozine yellow, bromophenol red, m-cresol purple, Eriochrome black T, safranine, luciferin, Eosin yellow, Brilliant green, Titan yellow, Victoria blue B, carmine, litmus, curcumin, anthocyanin, alizarin red S, alizarin yellow R, indigo carmine, nile blue A, orange yellow G, Eosin B, 3,3′,5,5′,-tetraiodophenol sulfone phenolphthalein, bromoxylenol blue, phenol blue, disperse orange 25, acridine orange, disperse orange 3, disperse red 1, bromopyrogallol red, diamine diphenyl sulfide maple, aminofluorescein, erythroviolet ammonium urea, 2,6-dichloroindophenol, sodium salts thereof, and mixtures thereof.

4. The device according to claim 1, wherein the sensing material includes a molecular barrier material selected from a group consisting of octadecanol, polyvinylpyrrolidone, polyvinyl formal, polyvinyl acetate resin, phenolic resin, epoxy resin, polybutene resin, polyethylene glycol, carbon black, carbon nanotubes, graphene, cellulose nanofibers, and silicone compounds.

5. A method for providing a food advertisement through color combinations based on food flavors, comprising the steps of:
sensing a food product with a colorimetric sensor array, which includes a plurality of sensing materials for sensing the food product, the plurality of sensing materials respectively reacting with at least one metabolic molecule of the food product to change from an initial color to an indication color; and
providing a label including a carrier and a plurality of color patches provided on the carrier, wherein the plurality of color patches corresponding to the indication colors is combined into a color pattern, so that the color pattern is visually associated with flavor information of the food product.

6. The method according to claim 5, wherein colors of the plurality of color patches are substantially equivalent to colors of the plurality of indication colors.
